(19) United States
(12) Patent Application Publication
(10) Pub. No.: US 2021/0278423 A1
(43) Pub. Date: Sep. 9, 2021

(54) FERRITIN ANALYSIS VIA LATERAL FLOW IMMUNOASSAY

(71) Applicant: George Arthur Donnelly, Corby Northants (GB)

(72) Inventor: George Arthur Donnelly, Corby Northants (GB)

(21) Appl. No.: 17/261,307

(22) PCT Filed: Jul. 16, 2019
(86) PCT No.: PCT/GB2019/051988

§ 371 Date: Jan. 19, 2021

(30) Foreign Application Priority Data
Jul. 16, 2018 (GB) 1811597.2
Jan. 21, 2019 (IE) S2019/0012

(51) Int. Cl. G01N 33/90; G01N 33/543; G01N 33/558; G01N 33/58

(52) U.S. Cl. CPC G01N 33/90; G01N 33/58; G01N 33/558; G01N 33/54306

(57) ABSTRACT

A lateral flow immunoassay (LFIA) strip comprises a sample pad (2) for receiving a sample and at least one conjugate pad (3) in operable contact with the sample pad. The at least one conjugate pad is loaded with a first antibody specific against ferritin and tagged with detectable label. A flow-through assay device for detecting ferritin levels in a sample is also provided comprising a sample pad (10) for receiving a sample and at least one conjugate pad (11) in operable contact with the sample pad. The at least one conjugate pad is loaded with a first antibody specific against ferritin and tagged with detectable label. An assay platform (12) is in operable contact with the at least one conjugate pad. The assay platform comprises a substrate having a test region onto which the first test antibody to the ferritin is immobilized. A method for measuring the level of ferritin in a sample is also provided. The sample may be a blood or saliva sample.

The present disclosure relates to devices and methods for the rapid detection of ferritin levels in a sample, in particular but not exclusively to assay devices for monitoring iron disorders and/or haemochromatosis severity e.g. by analysing saliva.

Hereditary haemochromatosis is a genetic condition characterised by iron overload, which untreated, can cause irreversible organ damage and result in chronic conditions including cirrhosis, diabetes, and arthritis. Diagnosis often comes after a patient has developed one of these conditions by which time a negative impact on quality-of-life has already occurred. The delay is due to clinical diagnosis requiring a full blood count confirmed by genetic screening; this is expensive and time-consuming for an overstretched, under-funded health service.

In general, cost and time-saving diagnosis measures are available with point of care tests (POCT) having been developed for many conditions. However, no POCT for hereditary haemochromatosis is currently available. This is in part due to the apparent rarity of hereditary haemochromatosis (640,000 patients in UK) as well as the development required for an accurate, rapid test for the key biomarker, serum ferritin (SF).

For adults, the healthy range of serum ferritin is 50-150 ng/ml. Hereditary haemochromatosis patients can see a serum ferritin level of >2000 ng/ml before diagnosis, with significant risk of cirrhosis. If diagnosed and treated before a serum ferritin level of >1000 ng/ml the risk is reduced to <1%. After initial treatment, maintenance therapy (venesection) is required every 2-4 months depending on the condition. Elevated serum ferritin levels of >300 ng/ml (male) and >200 ng/ml (female) should instigate this treatment, however, monitoring requires regular visits to GP/clinic with up to 2 weeks for test results to come through, during which time iron levels are constantly increasing.

The prevalence of hereditary haemochromatosis is predicted to be much higher than diagnosed (640k in the UK, 2.4 mn in the EU) but without a cost-effective diagnosis indicator (POCT) this population is not readily identified. This has a severe cost impact on the NHS in the UK, with £490 mn/year estimated preventable, as well as a massive quality-of-life impact on the undiagnosed hereditary haemochromatosis population.

Current hereditary haemochromatosis diagnostic or monitoring approaches include genetic screening, which is not cost-effective; and clinically-prescribed SF Enzyme Linked Immunosorbent Assay (ELISA) test at a central laboratory, (e.g. ADVIA_Centaur, £15.5k) or at the clinical point-of-care (GPs, clinics; e.g. Eurolyser Cube-S, £1.8k, 8 mins). However, such tests require operation by trained professionals with 1-2 weeks delay for results. Home-care blood tests (£25/test, pharmacy2u) are also available, but it can result in a 7-day delay for results.

An aim of the invention therefore is to provide a rapid, discreet, home-based point-of care-test for monitoring haemochromatosis severity, to enable cost-savings and improvement to the quality-of-life of haemochromatosis patients as well as other iron overload and deficient conditions.

According to a first aspect of the invention, there is provided a lateral flow immunoassay (LFIA) strip comprising a sample pad for receiving a sample. The lateral flow immunoassay strip may also comprise at least one conjugate pad in operable contact with the sample pad, the at least one conjugate pad loaded with a first antibody specific against ferritin and tagged with detectable label. The conjugate pad may contain a dried format of bioactive molecules in a salt sugar-matrix. The bioactive molecule may be the first antibody specific against ferritin and tagged with a detectable label.

The lateral flow immunoassay strip may also comprise an assay platform in operable contact with the at least one conjugate pad, the assay platform may comprise a substrate having a test region/line onto which the first test antibody to the ferritin is immobilized and optionally a control region/line onto which a control antibody directed to the first antibody is immobilized. Preferably the test line comprises the first a first antibody specific against ferritin without a detectable label

The lateral flow immune strip assay may also optionally comprise an absorbent pad in operable contact with the assay platform.

In a preferred embodiment of the invention, the lateral flow immunoassay strip comprises:
I. a sample pad for receiving a sample;
II. at least one conjugate pad in operable contact with the sample pad, the at least one conjugate pad loaded with a first antibody specific against ferritin and tagged with detectable label; preferably the conjugate pad contains a dried format of bioactive molecules in a salt sugar-matrix, wherein the bioactive molecules may be the first antibody specific against ferritin and tagged with detectable label;
III. an assay platform in operable contact with the at least one conjugate pad, the assay platform comprising a substrate having a test region/line onto which the first test antibody to the ferritin is immobilized and optionally a control region/line onto which a control antibody directed to the first antibody is immobilized, preferably the first test antibody in the test region is not labelled; and optionally,
IV. an absorbent pad in operable contact with the assay platform.

The sample pad, at least one conjugate pad, and the assay platform and optionally the absorbent pad preferably are arranged successively such that fluid flows from sample pad, to the at least one conjugate pad, to the assay platform and to the absorbent pad.

According to a second aspect of the invention, there is provided a flow-through assay device for detecting ferritin levels in a sample. The flow-through assay device may comprise a sample pad for receiving a sample.

The flow-through assay device may comprise at least one conjugate pad in operable contact with the sample pad, the at least one conjugate pad loaded with a first antibody specific against the ferritin and tagged with detectable label.

The flow-through assay device may comprise an assay platform in operable contact with at least one conjugate pad, the assay platform comprising a substrate having a test region onto which the first test antibody to the ferritin is immobilized and optionally a control region on to which a control antibody directed to the first antibody is immobilized.

The flow-through assay device may comprise an absorbent pad in operable contact with the assay platform.

In a preferred embodiment of the invention, there is provided a flow-through assay device for detecting ferritin levels in a sample, the device comprising:
I. a sample pad for receiving a sample;
II. at least one conjugate pad in operable contact with the sample pad, then at least one conjugate pad loaded with a first antibody specific against the ferritin and tagged with detectable label;
III. an assay platform in operable contact with the at least one conjugate pad, the assay platform comprising a substrate having a test region onto which the first test antibody to the ferritin is immobilized and optionally a control region onto which a control antibody directed to the first antibody is immobilized;
IV. optionally, an absorbent pad in operable contact with the assay platform.

The device of the invention may be used for analysing ferratin levels to assist with the maintenance and/or diagnosis of iron disorders, for example in respect of hemochromatosis, anaemia and iron overload.

According to a third aspect of the invention, there is provided a method for measuring the level of ferritin in a sample, wherein the method may comprise providing a flow through assay device that comprises a sample pad for receiving a sample.

The method may comprise at least one conjugate pad in operable contact with the sample pad, the at least one conjugate pad loaded with a first antibody specific against the ferritin and tagged with detectable label.

The method according to the invention may comprise an assay platform in operable contact with the at least one conjugate pad, the assay platform comprising a substrate having a test region onto which the first test antibody to the ferritin is immobilized and optionally a control region onto which a control antibody directed to the first antibody is immobilized.

The method according to the invention may comprise an absorbent pad in operable contact with the assay platform.

The method according to the invention may comprise contacting the conjugate pad with the sample and allowing the particles to flow to the test region.

The method according to the invention may comprise using a mobile test reader to scan the substrate to detect and quantify the level of ferritin on the test region; and optionally sending the results to a database.

In a preferred embodiment of the invention, there is provided a method for measuring the level of ferritin in a sample, said method comprising:
i) providing a flow-through assay device that comprises a sample pad for receiving a sample, at least one conjugate pad in operable contact with the sample pad, the at least one conjugate pad loaded with a first antibody specific against the ferritin and tagged with detectable label, an assay platform in operable contact with the at least one conjugate pad, the assay platform comprising a substrate having a test region on to which the first test antibody to the ferritin is immobilized and optionally a control region onto which a control antibody directed to the first antibody is immobilized; and optionally, an absorbent pad in operable contact with the assay platform;
ii) contacting the conjugate pad with the sample and allowing the particles to flow to the test region,
ii) using a mobile test reader to scan the substrate to detect and quantify the level of ferritin on the test region; and sending the results to a database.

According to a further aspect of the invention, there is provided a method for measuring the level of ferritin in a sample comprising;
i) providing a lateral flow immunoassay strip or device according to the invention;
ii) applying a sample to the sample pad, preferably a saliva sample;
iii) determining the intensity of labelled antibody bound to the test region/line;
iv) determining from the intensity of the label in iii) the level of ferritin in the sample.

The level of ferritin may be determined in step iv) by comparing the intensity level to known standard controls.

Preferably the method of the invention also comprises checking that label is observed on the control line, thereby confirming that the device is working.

The intensity of the label in step iii) may be determining by using a mobile test reader to scan the test line. The results observed in iv) may be stored in a database.

According to a yet further aspect the invention provides a kit for determining the level of ferritin in a sample, the kit comprising a device according to the invention, instruction to apply a sample of saliva to the sample pad and instructions to determine the level of ferritin from the intensity of label observed on the test line.

The term “antibody” is used here in its broadest sense refers to an immunoglobulin, or derivative or fragment or active fragment thereof, having an area on the surface or in a cavity which specifically binds to and is thereby defined as complementary with a particular spatial and polar organization of another molecule. The antibody can be monoclonal or polyclonal and can be prepared by techniques that are well known in the art such as, for example, immunization of a host and collection of sera or hybrid cell line technology, or recombinant technology. The term includes monoclonal antibodies, polyclonal antibodies, anti-idiotypic antibodies, synthetic antibodies, multi specific antibodies (e.g., bispecific antibodies) formed from at least two intact antibodies, and antibody fragments so long as they exhibit the desired binding activity. The antibodies can be chimeric antibodies, including humanized antibodies as described in Jones et al., Nature 321:522-525, 1986, Riechmann et al., Nature 332:323-329, 1988, Presta, Curr. Opin. Struct. Biol. 2:593-596, 1992, Vaswani and Hamilton, Ann. Allergy, Asthma &Immunol. 1:105-115, 1998, Harris, Biochem. Soc. Transactions 23:1035-1038, 1995, and Hurle and Gross, Curr. Opin. Biotech. 5:428-433, 1994.

Antibodies of any class or isotype (e.g., IgA, IgA1, IgA2, IgD, IgE, IgG, IgG1, IgG2, IgG3, IgG4,and IgM) can be used.

The term “antibody” also refers to any antibody fragment(s) that retain a functional antigen binding region. Examples of antibody fragments include Fab, Fab′, F(ab′)2, and Fv fragments; diabodies; linear antibodies; single-chain antibody molecules; and multi specific antibodies formed from antibody fragments, all of which are known in the art. Papain digestion of antibodies produces two identical antigen-binding fragments, called “Fab” fragments, each with a single antigen-binding site. Pepsin treatment yields an F(ab′)2 fragment that has two antigen-combining sites and is still capable of cross-linking antigen. Fab′ fragments differ from Fab fragments by the addition of a few residues at the carboxy terminus of the heavy chain CH1 domain including one or more cysteines from the antibody hinge region. F(ab′)2 antibody fragments are pairs of Fab′ fragments which have hinge cysteines between them. Other chemical couplings of antibody fragments are also known. “Fv” is the minimum antibody fragment which contains a complete antigen-binding site. Diabodies are described more fully in, for example, European Patent No. 404,097, International Patent Application WO 1993/01 161, Hudson et al., Nat. Med. 9:129-134, 2003, and Hollinger et al., PNAS USA90:6444-6448, 1993. Tria bodies and tetra bodies also are described in Hudson et al., Nat. Med. 9:129-134, 2003.

The term “antibody,” as used herein, also includes antibody substitutes or any natural, recombinant or synthetic molecule that specifically binds with high affinity to a particular target. Thus, the term “antibody” includes such synthetic antibodies or antibody substitutes such as aptamers, affibodies, affimers, avimers, aptides, and the like. Therefore, when describing the assay systems, devices and methods according to embodiments of the invention here, use of the term “antibody” for use as, for example, a reagent in the assay, indicates any of these alternatives can also be used.

The term “immobilized” (with respect to capture reagents or other reagents described herein) means that the migration of the capture molecule on the membrane or other surface on which it is immobilized (e.g., due to capillary flow of fluid such as the sample) or its escape from its immobilized location on the membrane is substantially impeded and, in certain embodiments, completely impeded. Methods for immobilizing the capture reagent are known in the art.

The term “sample” refers to any acquired material to be tested for the presence or amount of ferritin. Preferably, a sample is a fluid sample, preferably a blood or saliva sample. Preferably the sample is saliva. The term “sample” also includes material that has been collected from a subject and treated further, for example solubilized or diluted in a solvent suitable for testing.

The term ‘operable contact’ refers to direct or indirect contact (intervening component) between a first and second component of a test device whereby the contact allows fluid to flow, or communicate, from one component to another via gravity, capillary action or any other fluid flow.

The term ‘detectable label’ refers to any suitable label which may be a soluble label e.g. such as colorimetric, radioactive, enzymatic, luminescent or fluorescent label. The label may be a particle or particulate label, such as a particulate direct label, or a coloured particle label. The article or particle labels include but are not limited to colloidal gold label, latex particle label, nano particle label and quantum dot label.

The conjugate pad and/or the sample pad may be formed from a material through which the sample is capable of passing. For example, the conjugate pad and/or the sample pad may be formed from glass fibres. The conjugate pad and/or the sample pad may be formed from nitrocellulose, paper, nylon, or a synthetic nano-porous polymer.

The absorption pad and/or the wicking pad is a means for physically absorbing the sample which has chromatographically moved through the chromatography medium/lateral flow immune assay strip (e.g. via capillary action) and for removing unreacted substances. The absorption pad and/or wicking pad is located at the end of the lateral flow assay strip/device to control and promote movement of samples and reagents and acts as a pump and container for accommodating them. Commonly used absorption/wicking pads are formed of water-absorbing material such as cellulose filter paper, non-woven fabric, cloth or cellulose acetate.

The flow-through assay device or lateral flow immunoassay strip may be used in conjunction with a software application executable on a mobile test reader (i.e., a computing or processing device). This is intended to be construed broadly, and to cover personal and mobile computing devices as well as other intelligent devices comprising a processing means. The software application may be accessible by a user on any appropriate computing or processing device such as a mobile phone, wearable, watch, tablet, laptop or other personal electronic and/or computing device (such as a digital signal processor, a microcontroller, and an implementation in read only memory (ROM) or electronically erasable programmable read only memory (EEPROM), as non-limiting examples). The software application may be an assembly program.

The software application, including any saved data generated by the software application, may be stored locally on the mobile test reader, or remotely from the mobile test reader (e.g., in a cloud or other storage means, online or otherwise), and may be accessed via the internet or otherwise. The software application may be provided on a computer readable medium, which may be a physical computer readable medium, such as a disc or a memory device, or may be embodied as a transient signal. Such a transient signal may be a network download, including an internet download.

The mobile test reader may comprise or be in electronic communication with a camera. The camera may be used to capture one or more images of the substrate of the flow-through assay device after exposing the test region to the sample. Captured images may be stored locally on the mobile test reader or may be stored remotely from the mobile test reader.

The software application may be or comprise image capture and/or analysis software. The software application may be configured to use and/or control the camera to scan (i.e., capture images of) the substrate after exposing the test region to the sample. The software application maybe configured to read, analyse, interpret and quantify results obtained by the flow-through assay device by performing image analysis of the images of the substrate of the flow-through assay device captured by the camera. The software application may be configured to perform image analysis using one or more conventional image analysis techniques such as edge detection or colour detection, and/or by comparing the captured image or images to one or more reference images.

For example, the software application may be configured to identify if the first test antibody to ferritin has been immobilized on the test region by analysing the captured image or images to identify whether or not an appearance of the test region (e.g., colour, colour intensity, size, shape, pattern) on the captured image or images indicates immobilization of the first test antibody to ferritin on the test region. If the image analysis technique comprises comparing the captured images to one or more reference images, the reference images may be stored within or intrinsic to the software application, or may be images captured by the camera of a reference flow through assay representing, for example, either a positive test result (i.e., immobilization of the first test antibody to ferritin on the test region of the reference flow-through assay) or a negative test result.

### Example 1. Lateral Flow Immunoassay (LFIA), Antibody Capture

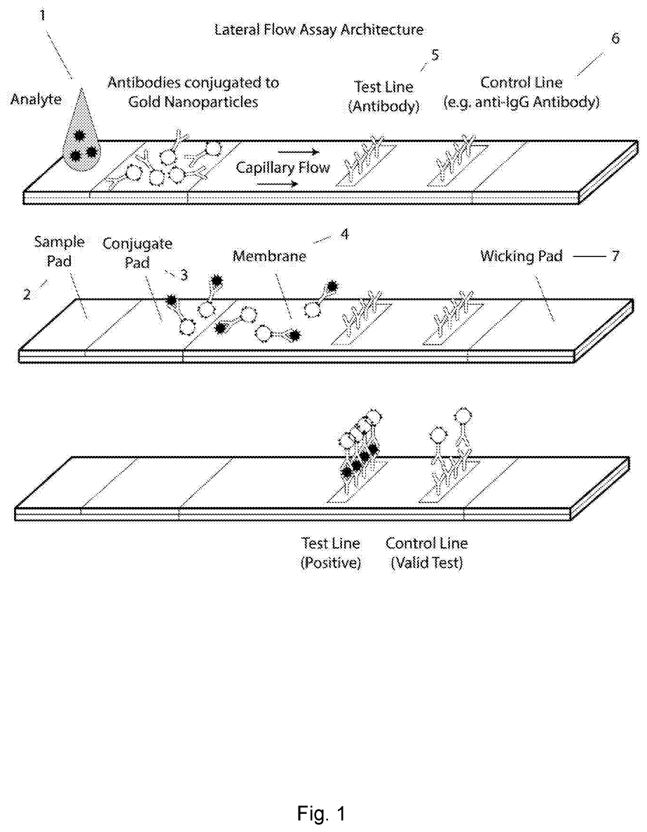

FIG. 1 shows a depiction of a LFIA arrangement according to the invention. Sample is loaded onto a sample pad, which is in operable contact/fluid communication with a conjugate pad that is loaded with conjugate antibody. A liquid, ferritin-containing sample disposed on the sample pad traverses from the sample pad to the conjugate pad where the conjugate antibody binds to ferritin in the sample. The ferritin-containing sample (now with ferritin/conjugate antibody complex) moves from the conjugate pad to the assay platform. The assay platform includes at least one test region having a test reagent immobilized thereon, the test reagent being specific to the ferritin. As the ferritin-containing sample traverses across the assay platform, ferritin of the ferritin/conjugate antibody complex binds to the test reagent. The assay platform also includes a control region which has an antibody that is directed to conjugate antibody. This assists in determining whether the assay developed properly, and/or assists in calibrating the signal of the test region(s). The LFIA may also include an absorbent pad, which helps drive the flow of ferritin containing sample via capillary action.

In the context of lateral flow immunoassay devices, these are typically constructed of a solid support that provides lateral flow of a sample through the assay platform when a sample is applied to a sample pad that is inoperable contact with the assay platform. The sample pad and the assay platform are typically constructed of a material such as nitrocellulose, glass fibre, paper, nylon, or a synthetic nano porous polymer. Suitable materials are well known in the art and are described, for example, in U.S. Pat. No. 7,256,053 to Hu, U.S. Pat. No. 7,214,417 to Lee et al., U.S. Pat. No. 7,238,538 to Freitag et al., U.S. Pat. No. 7,238,322 to Wang et al., U.S. Pat. No. 7,229,839 to Thayer et al., U.S. Pat. No. 7,226,793 to Jerome et al., RE39,664 to Gordon et al., U.S. Pat. No. 7,205,159 to Cole et al., U.S. Pat. No. 7,189,522 to Esfandiari, U.S. Pat. No. 7,186,566 to Qian, U.S. Pat. No. 7,166,208 to Zweig, U.S. Pat. No. 7,144,742 to Boehringer et al., U.S. Pat. No. 7,132,078 to Rawson et al., U.S. Pat. No. 7,097,983 to Markovskyet al., U.S. Pat. No. 7,090,803 to Gould et al., U.S. Pat. No. 7,045,342 to Nazareth et al., U.S. Pat. No. 7,030,210 to Cleaver et al., U.S. Pat. No. 6,981,522 to O'Connor et al., U.S. Pat. No. 6,924,153 to Boehringer et al., U.S. Pat. No. 6,849,414 to Guan et al., U.S. Pat. No. 6,844,200 to Brock, U.S. Pat. No. 6,841,159 to Simonson, U.S. Pat. No. 6,767,714 to Nazareth et al., U.S. Pat. No. 6,699,722 to Bauer et al., U.S. Pat. No. 6,656,744 to Pronovost et al., U.S. Pat. No. 6,528,323 to Thayer et al., U.S. Pat. No. 6,297,020 to Brock, U.S. Pat. No. 6,140,134 to Rittenburg, U.S. Pat. No. 6,136,610 to Polito et al., U.S. Pat. No. 5,965,458 to Kouvonen et al., U.S. Pat. No. 5,712,170 to Kouvanen et al., U.S. Pat. No. 4,956,302 to Gordon et al., and U.S. Pat. No. 4,943,522 to Eisinger et al., all of which are incorporated herein by this reference. The use of such devices for the performance of sandwich immunoassays is also well known in the art, and is described, for example, in U.S. Pat. No. 7,141 ,436 to Gatto-Menking et al. and U.S. Pat. No. 6,017,767 to Chandler, U.S. Pat. No. 6,372,516 to Ming Sun, all of which are incorporated herein by this reference.

As indicated above, a ferritin-containing sample traverses the assay platform by way of capillary action. As the sample moves across the assay platform ferritin in the sample encounter different biomolecules, such as antibodies, that bind to the ferritin. Materials from which the assay platform can be made typically include, but are not limited to, nitrocellulose, glass fibre, paper, nylon, or a synthetic sample via capillary action.

LFA reproducibility is not only influenced by design and manufacturing, but also by the components used in the assembly of the test. The assay platform membrane has a significant impact on the performance of the test results. Nitrocellulose membranes are manufactured by dissolving the raw materials in a mixture of organic solvents and water, pouring this casting mix onto a solid belt-like support, and evaporating the solvents under controlled conditions of temperature, humidity, and belt speed within the membrane manufacturing machine.

Using new membrane formulations and improved manufacturing conditions various Variables of the membrane can be controlled according to techniques known in the art. Such variables include:

i. Flow Rate of Membrane—This is determined empirically and will vary according to the viscosity of the sample used. Data for the flow rates of specific membranes with specific sample types are supplied by the manufacturer.

ii. Membrane Porosity—This describes the fraction of the membrane that is air (e.g., a membrane with a porosity of 0.7 is 70% air), and will have an impact on the flow rate of the membrane.

iii. Membrane Capacity—By definition, this is the amount of volume of sample that a given membrane can hold, and is determined as a factor of the length (L), width (W), thickness (T), and porosity (P) of the membrane: L×W×T×P=Membrane Capacity. A second important calculation is the determination of the amount of antibody that can be bound per unit area of membrane (pertaining to the capture and control lines). This calculation involves the following variables. Dimensions of representative capture antibody line: 0.1 cm×0.8 cm=0.08cm2. Binding capacity of membrane used for capture antibody (obtained from the membrane manufacturer).

In an embodiment, the conjugate pad comprises an absorbed but not immobilized conjugate comprising a conjugate reagent (e.g. antibody) specific for the ferritin and conjugated directly to detectable marker, such as a gold nanoparticle. The conjugate reagent can be loaded onto the conjugate pad using an aqueous conjugate antibody solution. The conjugate antibody solution comprises the conjugate antibody and other components provided for solution stability, pH regulation, and the like. Examples of such ancillary components include buffers, salts, preservatives, etc. Specific examples include BSA, sucrose, trehalose, tween-20, PEG, water, HEPES, Polyvinyl pyrolidone (PVP), and the like. In an embodiment, the conjugate antibody solution is applied to the conjugate pad and allowed to dry for a period of time (e.g., 0.5, 0.7, 1, 1.5 hr) at a specified temperature (e.g., 23, 25, 30, 35, 37, 40° C.). By such application or an equivalent method, the conjugate is loaded onto the conjugate pad. In this regard, the conjugate is not immobilized on the conjugate pad, and can be carried from the conjugate pad via an assay solution, such as along a capillary flow path, into the porous membrane.

It will be appreciated that optional features applicable to one aspect or embodiment of the invention can be used in any combination, and in any number. Moreover, they can also be used with any of the other aspects or embodiments of the invention in any combination and in any number. This includes, but is not limited to, the dependent claims from any claim being used as dependent claims for any other claims of this application.

Embodiments of the present invention will now be further described, purely by way of example, with reference to the accompanying drawings, in which

FIG. 1 shows a schematic diagram of a test strip based on lateral flow immunoassay according to the invention; and

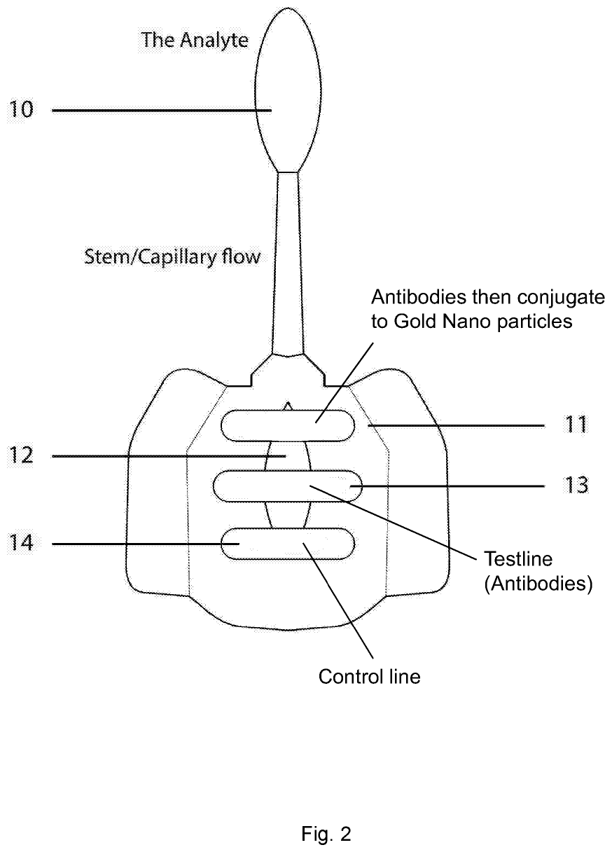

FIG. 2 shows a schematic diagram of a flow-through assay device according to the invention for detecting ferritin levels in a sample.

The invention now being generally described, it will be more readily understood by reference to the following examples which are included merely for purpose of illustration of certain aspects and embodiments of the present invention and are not intended to limit the invention in anyway.

The method of the present disclosure uses lateral-flow assay to provide a sensitive tool for detecting ferritin in a sample.

The sample 1 containing the analyte (ferritin) is applied to the sample pad 2.

From the sample pad 2, the sample travels to the conjugate pad 3, where the sample mixes with a first antibody specific against ferritin to form analyte complexes. Because the conjugate pad 3 is in fluid communication with the membrane 4, the analyte complexes then migrate from the conjugate pad 4 to the test line (region) 5 and control line (region) 6.

The first test antibody to the ferritin is immobilized at the test line (region) 5 and optionally the control antibody directed to the first antibody is immobilized at the control line (region) 6.

The sample then optionally enters the wicking pad 7 which acts as a waste container. The flow-through assay device containing the sample pad 10 receives the sample containing the analyte.

From the sample pad 10, the sample travels to the conjugate pad 11 which is in operable contact with the sample pad 10. The conjugate pad 11 is loaded with a first antibody specific against the ferritin and tagged with a detectable label. The assay platform 12 is in operable contact with the conjugate pad 11 and the assay platform 12 comprises a substrate having a test line (region) 13 onto which the first test antibody to the ferritin is immobilized and optionally a control line (region) 14 on to which a control antibody directed to the first antibody is immobilized.

Wherein a number of embodiments of the present invention have been shown and described herein in the present context, such embodiments are provided by way of example only, and not of limitation. Numerous variations, changes and substitutions will occur to those of skill in the art without materially departing from the invention herein.

To summarise, the invention provides a lateral flow device for detecting the level of ferritin in a sample. Preferably the sample is saliva.

In an embodiment the invention provides a lateral flow device comprising a sample pad 2, a conjugate pad 3, a test line 5, a control line 6 and optionally a wicking pad 6. The sample pad 2, onto which a sample to be tested is placed, is in fluid communication with the conjugate pad 3. The conjugate pad contains first antibodies to ferritin, the antibodies are free in the conjugate pad, that is they are not bound to the device. The first antibodies are labelled, for example with gold nanoparticles. The conjugate pad 3 is in fluid communication with the test line 5 and/or the control line 6. The test line 5 comprises antibodies bound to the lateral flow device which are directed to ferritin bound to the first antibodies. The antibodies bound to the test line may be the same as the first antibodies without the label. The test line is in fluid communication with the conjugate pad and/or the control line. The control line 6 comprises antibodies bound to the lateral flow device which are directed to the first antibody, preferably to a part of the first antibody that is masked if the first antibody is bound to ferritin. The control line is in fluid communication with the test line and/or the conjugate pad. The test line and control line may be referred to together as the assay platform. The wicking pad 6 is configured to absorb any sample that is not bound to the test or control line, and is in fluid communication with the control line and/or the test line.

In use a sample, preferably a saliva sample is placed on the sample pad 2 on the lateral flow device of the invention. The sample then moves through the device to the conjugate pad 3 wherein ferritin in the sample is bound by the first antibodies. The sample and antibodies from the conjugate pad then move to the test and/or control line. Ferritin bound to the first antibodies bind to antibodies in the test line and the first antibodies are immobilised at the test line. Unbound first antibodies (that is, not bound to ferritin) bind to antibodies in the control line and are immobilised there. Antibodies immobilised to the control and test line are visualised using the label on the first antibodies. Binding to the control line shows the lateral device is working. Binding to the test line shows the presence, and preferably the level, of ferritin in the sample. Any unbound sample may move through the device to the wicking pad. The sample moves through the device by wicking.

Aspects/embodiments of the invention may be implemented in a hand-held point of care (POC) device. The device may comprise a casing to support the lateral flow assay and analyte. This can be converted to a quantatative reading of ferratin, e.g. when combined with using an electronic or mobile device. Once the saliva/blood is placed on the analyte and passes through the lateral flow assay, the ferratin level can be converted to a reading, e.g. by a processor, which is viewable via a mobile device.

The advantages of the invention include the following: there is currently no hand-held point of care (POC) device to establish ferratin levels outside of a laboratory environment, and as such no method for a Hemochromatosis patient and/or iron disorder patient such as; anaemia or iron overload to monitor their own ferratin levels. The provision of a POC device will allow a patient with an iron disorder to monitor their own ferratin levels, allowing them to either; donate to a local blood bank and/or adjust their food intake to maintain a healthy level. In addition to this; removal of the patient from the norms of the NHS protocols will free up valuable time to be utilised elsewhere, notwithstanding the savings to the NHS in time, equipment; together with the cost to the patient, parking, time off work, and the employer to assist with their daily homecare outside of the scope of the NHS care system. Further, enhancing the quality of life for the patient, assisting GPs with a ferratin result reading in a timely manner (e.g. within about 3 mins), without the need for routine blood tests and laboratory testing.

1. A lateral flow immunoassay (LFIA) strip, comprising: a sample pad for receiving a sample; at least one conjugate pad in operable contact with the sample pad, the at least one conjugate pad loaded with a first antibody specific against ferritin and tagged with detectable label; wherein the conjugate pad optionally contains a dried format of bioactive molecules in a salt sugar-matrix; an assay platform in operable contact with the at least one conjugate pad, the assay platform comprising a substrate having a test region onto which the first test antibody to ferritin is immobilized and optionally a control region onto which a control antibody directed to the first antibody is immobilized; and optionally, an absorbent pad in operable contact with the assay platform.

2. A flow-through assay device for detecting ferritin levels in a sample, the device comprising: a sample pad for receiving a sample; at least one conjugate pad in operable contact with the sample pad, wherein the at least one conjugate pad is loaded with a first antibody specific against ferritin and tagged with detectable label; an assay platform in operable contact with the at least one conjugate pad, the assay platform comprising a substrate having a test region onto which the first test antibody to the ferritin is immobilized and optionally a control region onto which a control antibody directed to the first antibody is immobilized; optionally, an absorbent pad in operable contact with the assay platform.

3. A method for measuring the level of ferritin in a sample, said method comprising: i) providing a flow-through assay device that comprises a sample pad for receiving a sample, at least one conjugate pad in operable contact with the sample pad, the at least one conjugate pad loaded with a first antibody specific against the ferritin and tagged with detectable label, an assay platform in operable contact with the at least one conjugate pad, the assay platform comprising a substrate having a test region on to which the first test antibody to the ferritin is immobilized and optionally a control region onto which a control antibody directed to the first antibody is immobilized; and optionally, an absorbent pad in operable contact with the assay platform;
(ii) contacting the conjugate pad with the sample and allowing the particles to flow to the test region,
iii) using a mobile test reader to scan the results and detect and quantify the level of ferritin; and sending the results to a database.

4. A method for measuring the level of ferritin in a sample comprising;
i) providing a lateral flow immunoassay strip or device according to the invention;
ii) applying a sample to the sample pad, preferably a saliva sample;
iii) determining the intensity of labelled antibody bound to the test region/line;
iv) determining from the intensity of the label in iii) the level of ferritin in the sample.

5. A method according to claim 3, wherein the sample is a blood or saliva sample.

6. A method according to claim 3, wherein the sample is a saliva sample.

7. (canceled)

8. A method of monitoring and/or diagnosing an iron disorder in a subject, the method comprising;
i) providing a sample of saliva from the subject;
ii) performing the method of claim 4;
iii) determining from the level of ferritin observed in the sample if the subject has an iron disorder or monitoring the progression of a subject with an iron disorder.

9. The method of claim 8 wherein in step iii) the level of ferritin observed is compared to the level observed in a previous sample from the same subject.

10. The method of claim 8 wherein the iron disorder is hemochromatosis, anaemia or an iron overload.

11. A lateral flow immunoassay strip according to claim 1, wherein the sample is a blood or saliva sample.

12. A kit for determining the level of ferritin in a sample, the kit comprising a lateral flow immunoassay strip according to claim 1, instructions to apply a sample of saliva to the sample pad and instructions to determine the level of ferritin from the intensity of label observed on the test line.

13. A flow-through assay device according to claim 2, wherein the sample is a blood or saliva sample.

14. A kit for determining the level of ferritin in a sample, the kit comprising a flow-through assay device according to claim 2, instructions to apply a sample of saliva to the sample pad and instructions to determine the level of ferritin from the intensity of label observed on the test line.

15. A method according to claim 3, wherein the sample is a blood or saliva sample.
